(19) United States
(12) Patent Application Publication
(10) Pub. No.: US 2026/0123835 A1
(43) Pub. Date: May 7, 2026

(54) MULTIMODAL DEVICE FOR SPECTRAL AND OCT ACQUISITIONS

(71) Applicant: ZILIA INC., Québec (CA)

(72) Inventors: Nicolas LAPOINTE, Québec (CA); Damon DEPAOLI, Arlington, MA (US); Dominic SAUVAGEAU, Québec (CA)

(21) Appl. No.: 19/118,138

(22) PCT Filed: Nov. 15, 2023
(86) PCT No.: PCT/CA2023/051534

§ 371 Date: Apr. 3, 2025

Related U.S. Application Data
(60) Provisional application No. 63/383,766, filed on Nov. 15, 2022.

(51) Int. Cl. A61B 3/12; A61B 3/00; A61B 3/10

(52) U.S. Cl. CPC A61B 3/1225; A61B 3/0008; A61B 3/102

(57) ABSTRACT

A multimodal device for performing an assessment of the fundus of a patient's eye is provided. The multimodal device includes an imaging module producing an image representative of a first region of the fundus, a spectroscopy module producing a spectral analysis of a second region of the fundus and an OCT module producing depth-resolved information on a third region of the fundus. The second region is located within the first region, and the third region is at least partially located within the first region. Electromagnetic radiation travels from the fundus to the imaging module, spectral module and OCT module along an imaging path, a spectral path and an OCT path which a common light path segment leading to the fundus of the patient's eye.

### TECHNICAL FIELD

The technical field generally relates to imaging techniques and, more specifically, to devices and methods combining spectral information and optical coherence tomography (OCT), in particular of the eye fundus.

### BACKGROUND

Ocular oximetry, that is, the measurement of the degree of oxygen saturation of blood in tissues of the eye, is a useful non-invasive tool with widespread medical and health monitoring applications. Indeed, measurement of oxygen saturation in biological tissues can provide valuable information on metabolism, responses to stress, the pathophysiology of different illnesses and conditions or the efficacy of administered treatments.

Spectroreflectometric systems can be used to provide oximetry measurements or other information from the fundus of a patient's eye through a spectral analysis of light resulting from the interaction of illumination light with the fundus medium, or features in the eye fundus. For optimal use in the field, oximeters or similar devices and systems should preferably be efficient, simple to use and easy to manufacture. Low cost and miniaturisation of such equipment are also factors of interest.

International patent application No. WO2019/109186 (PRIOR ART) teaches a spectroreflectometric system which provides a combined imaging and spectral analysis of the eye fundus. Advantageously, the disclosed system provides both a 2D image of the eye fundus as well as a spectral analysis at an analysis area on the fundus. A pointer mode allows for a user to visualize the position of the analysis area within the 2D image.

In practice, fundus imagery based on the acquired 2D RGB images has been used for years by eye-care professionals to identify suspicious retinal features, for example associated with pathologies. For more specific information and metabolism-related information, additional tests are required, typically using different platforms. Optical coherence tomography (OCT) is typically used to show internal structural details, including in the area where the abnormal occurrence was identified. In addition to these techniques, diffuse reflectance spectroscopy enables the evaluation of specific biomarkers from a specific location, these biomarkers providing indications of the medical state of the patient.

There remains a need for techniques acquiring multimodal information from a specific region of the eye fundus to provide a complete assessment of the state of the region, from oxygen saturation, retina thickness and blood flow.

### SUMMARY

In accordance with one aspect, there is provided a multimodal device for performing an assessment of the fundus of a patient's eye, the multimodal device comprising:
an imaging module comprising an imaging sensor having an angular field of view encompassing a first region of the fundus, the imaging sensor configured to receive electromagnetic radiation from said first region through an imaging path and to produce therefrom an image representative of said first region;
a spectroscopy module comprising a spectral analyzer having an angular field of view encompassing a second region of the fundus, the second region being located within the first region, the spectral analyzer configured to receive electromagnetic radiation from the second region of the fundus through a spectral path and to produce therefrom a spectral analysis of said second region;
an OCT module comprising an OCT detector having an angular field of view encompassing a third region of the fundus, the third region being at least partially located within the first region, the OCT detector configured to receive electromagnetic radiation from the third region of the fundus through an OCT path and to produce therefrom depth-resolved information on said third region;
wherein the imaging path, the spectral path and the OCT path have a common light path segment leading to the fundus of the patient's eye.

In some implementations, the multimodal device further comprises an illumination module comprising a non-coherent light source generating illumination light and optically coupled to the common light path segment for illuminating the first region of the fundus of the patient's eye with said illuminating light.

In some implementations, the spectroscopy module comprises an excitation light source generating an excitation light beam coupled to the spectral path for projecting on the second region of the fundus of the patient's eye.

In some implementations, the multimodal device further comprises a pointer light source generating a pointer light beam coupled to the spectral path for projecting on the second region of the fundus of the patient's eye.

In some implementations, the spectral analysis produced by the spectral module comprises a spectroreflectometric analysis and/or a photoluminescence analysis.

In some implementations, the OCT module further comprises:
an OCT light source configured to generate an OCT light beam having a short coherence length;
a reference arm; and
an OCT coupler configured to divide the OCT light beam into a first portion and a second portion and optically coupled to the OCT light path and to the reference arm;
whereby the first portion of the OCT light beam is coupled into the OCT light path for projection on the third region of the fundus of the patient's eye, and the second portion of the OCT light beam is coupled into the reference arm.

In some implementations, the OCT module has a Time-Domain OCT configuration. In other implementations, the OCT module has a Spectral Domain OCT configuration.

In some implementations, the multimodal device further comprises a scanning module provided along the OCT path and configured to move the first portion of the OCT light beam across the fundus of the patient's eye, thereby defining the third region.

In some implementations, the spectral path and the OCT path have a shared spectral and OCT segment, and the scanning module is provided within the shared spectral and OCT segment.

In some implementations, the multimodal device comprises:
a primary beamsplitter connecting the imaging path and the shared spectral and OCT segment, the common light path segment extending between the primary beamsplitter and the fundus of the patient's eye; and
a secondary beamsplitter connecting the spectral path and the OCT path, the shared spectral and OCT segment extending between the secondary beamsplitter and the primary beamsplitter.

In some implementations, the multimodal device comprises:
a primary beamsplitter coupling the spectral path and the imaging path, the common light path segment extending between the primary beamsplitter and the fundus of the patient's eye;
a secondary beamsplitter coupling the OCT light path to the imaging light path downstream of the primary beamsplitter.

In some implementations, the multimodal device further comprises a controller generating control signals for controlling active components of the imaging module, spectral module, OCT module, illumination module and scanning module. The controller may be configured to operate the multimodal device to:
a) acquire the image representative of said first region using the imaging module;
b) identifying the second region and third region based on said image;
c) acquire the spectral analysis of said second region;
d) acquire the depth-resolved information on the third region.

In some implementations, the controller is configured to operate the multimodal device in:
an illumination mode wherein the controller operates the illumination light source to project said illumination light towards the patient's eye, the imaging device to obtain the image representative of said first region of the fundus and the spectral analyser to obtain said spectral analysis of said second region of the fundus; and
an OCT mode wherein the controller operates the OCT light source, OCT detector and scanning module to obtain the depth-resolved image of the third region of the fundus.

Embodiments described here allow the combination of non-invasive OCT imaging, color fundus imaging and spectral analysis from a targeted region of the eye fundus. In some implementations, such a combination would allow for a variety of applications including but not limited to diagnosis of diseases of the eye.

Other features and advantages will be better understood upon a reading of preferred embodiments with reference to the appended drawings.

### BRIEF DESCRIPTION OF THE DRAWINGS

FIG. 1A is a schematized representations of a multimodal device as described herein; FIG. 1B is a perspective view of a multimodal device housed in a casing according to one embodiment.

FIGS. 2A and 2B illustrate different representations of the first, second and third regions of the fundus od a patient's eye according to one aspect.

FIGS. 3A and 3B are schematic representations of a multimodal device according to a first configuration.

FIGS. 4A and 4B are schematic representations of a multimodal device according to a second configuration.

FIGS. 5A and 5B show an image of the fundus acquired by an imaging module, and the selection of a second region for spectral analysis thereon; FIG. 5C show a spectrum acquired using the spectral module; and FIG. 5D shows a cross-sectional image acquired using the OCT module.

FIG. 6A shows an image of a pointer light beam obtained in a pointer mode; FIG. 6B shows an image of the first region of the fundus and FIG. 6C shows a spectrum of the second region of the fundus corresponding to the area targeted by the pointer light beam; FIG. 6D shows a timing diagram for sending control signals to the pointer light source, the non-coherent light source, the imaging sensor and the spectral analyzer.

FIG. 7 shows a timing diagram for sending control signals to the non-coherent source, the imaging sensor and the OCT light source.

### DETAILED DESCRIPTION

In the following description, similar features in the drawings have been given similar reference numerals. In order not to unduly encumber the figures, some elements may not be indicated on some figures if they were already mentioned in preceding figures. It should also be understood herein that the elements of the drawings are not necessarily drawn to scale and that the emphasis is instead being placed upon clearly illustrating the elements and structures of the present embodiments.

The terms “a”, “an” and “one” are defined herein to mean “at least one”, that is, these terms do not exclude a plural number of items, unless stated otherwise. Terms such as “substantially”, “generally” and “about”, that modify a value, condition or characteristic of a feature of an exemplary embodiment, should be understood to mean that the value, condition or characteristic is defined within tolerances that are acceptable for the proper operation of this exemplary embodiment for its intended application.

Unless stated otherwise, the terms “connected” and “coupled”, and derivatives and variants thereof, refer herein to any structural or functional connection or coupling, either direct or indirect, between two or more elements. For example, the connection or coupling between the elements may be mechanical, optical, electrical, logical, or any combination thereof.

In the present description, the terms “light” and “optical”, and variants and derivatives thereof, are used to refer to radiation in any appropriate region of the electromagnetic spectrum. The terms “light” and “optical” are therefore not limited to visible light, but can also include, without being limited to, the infrared or ultraviolet regions of the electromagnetic spectrum. Also, the skilled person will appreciate that the definition of the ultraviolet, visible and infrared ranges in terms of spectral ranges, as well as the dividing lines between them, may vary depending on the technical field or the definitions under consideration, and are not meant to limit the scope of applications of the present techniques.

The present description generally relates to a multimodal device and method for performing an assessment of the fundus of a patient's eye, by combining imaging, spectral analysis and OCT information about the retina or other medium.

In some implementations, the disclosed device and method enable a comprehensive assessment of a specific region of the fundus of the eye, which can aid clinicians in the identification of abnormal occurrences.

Spectral acquisitions refer to the use of light or other electromagnetic waves to analyze the properties of a medium. Light is typically projected towards the medium and the interactions of the light wavefront with the medium leads to the generation of return light having optical properties affected by the medium. A spectral analysis of the return light, that is, an analysis of the properties of the return light as a function of its wavelength profile, is used to obtain or deduce information about the medium and its composition.

The acquired information can be used in ophthalmologic contexts to sense oxygen levels in the fundus of the eye of a patient. By way of example, oxygen levels are assessed through the presence of oxyhemoglobin which has a characteristic light absorbance pattern. Similarly, the concentration of deoxyhemoglobin, and carboxyhemoglobin (related to the levels of carbon dioxide present) can be determined based on their respective light absorbance patterns. These parameters and their regulation are indicative of metabolism, responses to stress and stimuli and, potentially, pathophysiology. It will however be understood that other molecules and phenomena may also be studied, such as for example fluorescence, since they lead to alterations in the spectral profiles of reflected light. It will also be understood that the spectral analysis may be performed for different regions of the fundus of the patient's eye or on features present on the fundus. In other implementations, the spectral analysis may be performed on other portions of the eye such as the conjunctiva.

Optical coherence tomography (OCT) is an optical imaging technique enabling depth-resolved acquisition of images using a low coherence interferometer system. OCT, for example, enables eye-professionals to visualize three-dimensional structures of the fundus of the eye and to measure the thickness of each layer of the retina. This information can be used to aid in the detection and diagnosis of retinal diseases and conditions, such as glaucoma, age-related macular degeneration (AMD) and diabetic retinopathy. OCT is nowadays a widespread diagnostic device used in optometry and ophthalmology contexts. While the structural information is valuable, the ability to understand the functional state of a tissue is missing.

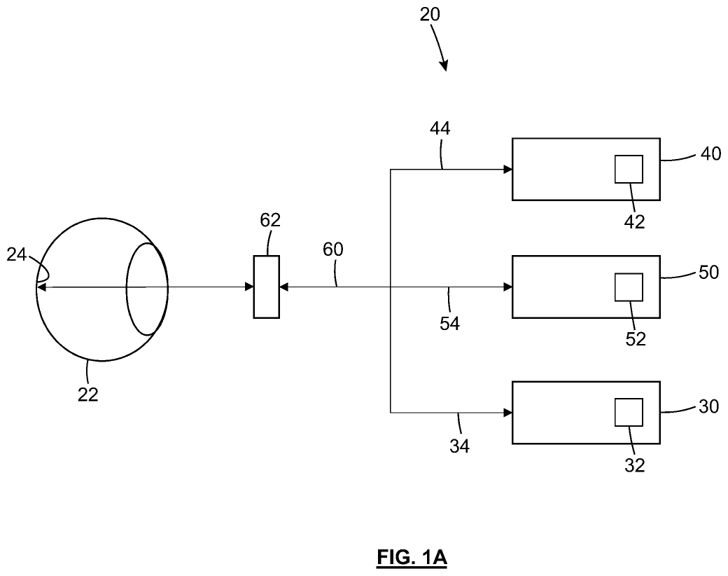

Referring to FIGS. 1A and 1B, a multimodal device 20 for performing an assessment of the fundus 24 of a patient's eye 22 according to some implementations is shown.

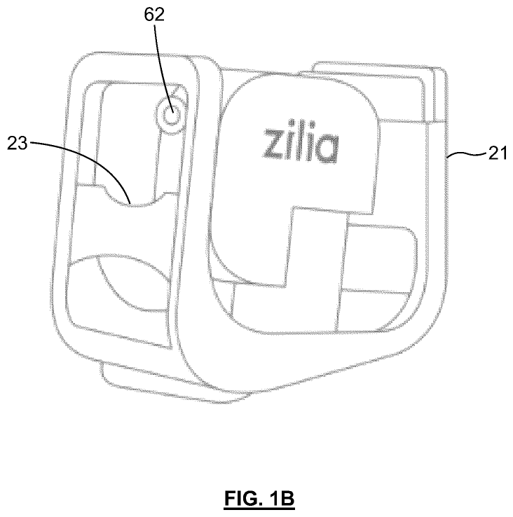

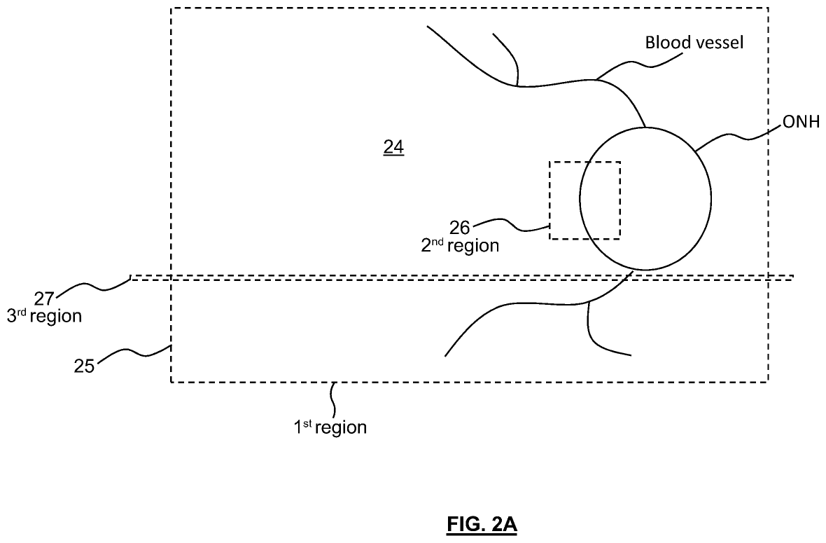

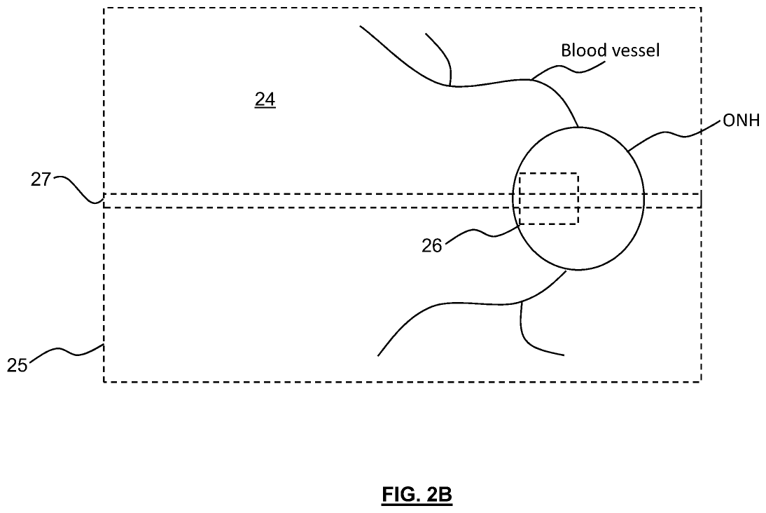

The multimodal device 20 generally includes an imaging module 30. Referring additionally to FIGS. 2A and 2B, the imaging module 30 comprises an imaging sensor 32 having an angular field of view encompassing a first region 25 of the fundus 24 of the patient's eye 22. The multimodal device 20 further includes a spectroscopy module 40 comprising a spectral analyzer 42 having an angular field of view encompassing a second region 26 of the fundus 24. The second region 26 is located within the first region 25. The multimodal device 20 further includes an OCT module 50. The OCT module 50 comprises an OCT detector 52 having an angular field of view encompassing a third region 27 of the fundus 24. The third region 27 is at least partially located within the first region 25. Both the second region 26, covered by the spectroscopy module 40, and the third region 27, covered by the OCT module 50, can be displaced, for example via displacement of a scanning module and/or a beamsplitter, as explained further below. The image of the first region, in addition to providing information from the fundus, may also serve as a reference for selecting the second and/or the third region. By way of example, selection of the second or third region can be based on a visual anomaly identified on the first image or on routine protocol that requires targeting specific regions of the eye fundus such as the optic nerve head (ONH), as depicted on FIG. 2B. In one example of implementation, the first region may be centered on the fundus region that contains the optic nerve head, while the second region acquires spectroreflectometric measurements from the temporal section of the ONH, and the third region performs a B-Scan of the retina and the ONH. The sum of this information can provide global morphological and structural information to the clinician about a given location of the eye fundus.

As schematically illustrated in FIG. 1A, the multimodal device 20 is preferably configured to guide light along an imaging path 34, a spectral path 44 and an OCT path 54, optically coupling the fundus 24 of the patient's eye to the imaging module 30, the spectroscopy module 40 and the OCT module 50, respectively. All of the modules and components of the multimodal device 20 may be integrated together within a casing 21 or a casing assembly adapted for use in a clinical setting, as for example illustrated in FIG. 1B. The casing 21 may have any suitable form and shape and may be sized to be placed on a table or other equivalent support structure, or in other variants may be designed to be handheld. The imaging path 34, the spectral path 44 and the OCT path 54 all have a common light path segment 60 leading to the fundus 24 of the patient's eye 22. The common light path segment 60 may include an objective lens 62 or other optical components or an ensemble of optical components providing a light interface between the inside and outside of the casing 21. The casing 21 may include a head support rest 23 or other structure designed for the patient to position his eye at a location aligned with the common light path segment, as well known in the art. The multimodal device 20 may further include one or more alignment mechanisms as also well known in the art.

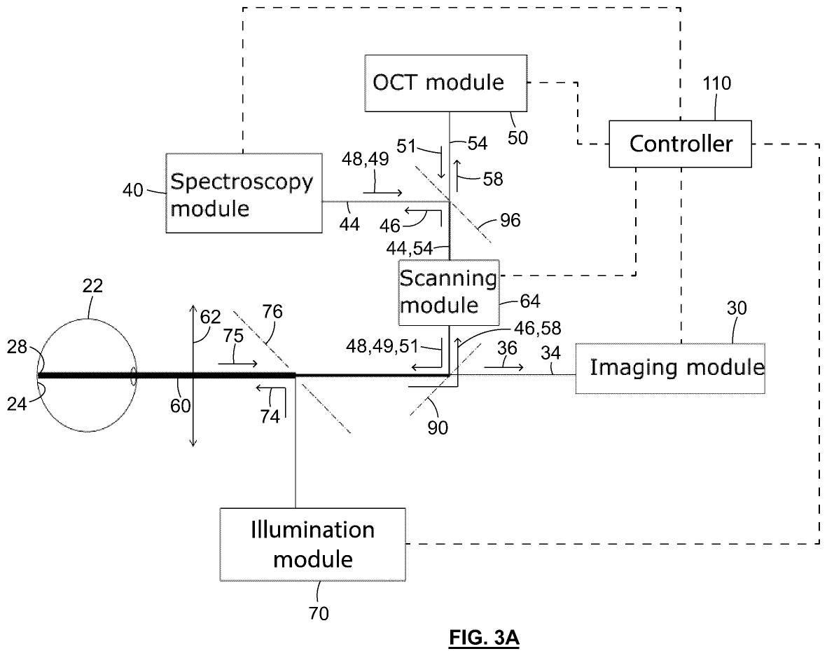

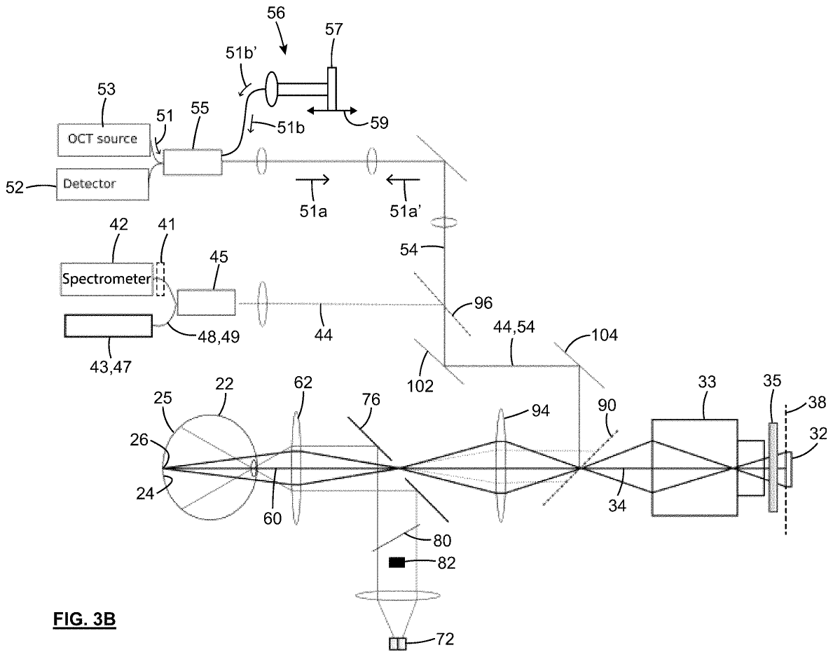

Referring to FIGS. 3A and 3B, a configuration of a multimodal device 20 according to one embodiment is shown, respectively as a high-level schematic representation and a more detailed example of implementations.

### Imaging Module

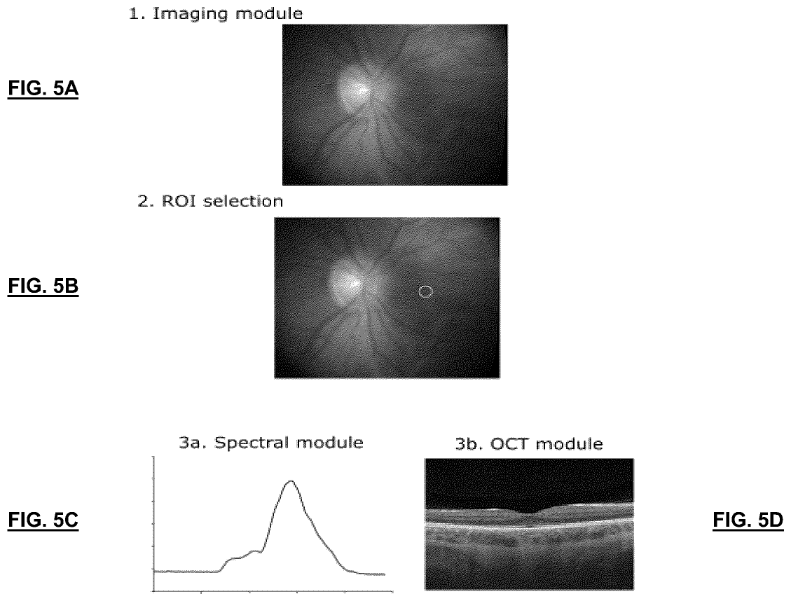

As mentioned above, the imaging module 30 comprises an imaging sensor 32 configured to receive electromagnetic radiation from the first region 25 through the imaging path 34 and to produce therefrom a 2D image representative of said first region 25 (see for example FIG. 5A). In some implementations, the imaging sensor 32 may be embodied by a CCD or CMOS sensor, or any surface that is sensitive and converts light intensity or energy into a useful signal. The imaging sensor 32 may include or be in communication with a processor, computer, circuit or any other hardware component or ensemble of hardware components programmed with instructions for constructing, storing and/or displaying the images acquired by the imaging sensor 32. An integrated or separate display may be provided to allow the viewing of the resulting images by an operator or user of the multimodal device 20.

The multimodal device 20 further includes an illumination module 70 including a non-coherent light source 72 generating illumination light 74 and optically coupled to the common light path segment 60 for illuminating the first region 25 of the fundus 24 of the patient's eye 22 with this illuminating light 74. The non-coherent source 72 of electromagnetic radiation is operationally coupled to the imaging module 30. The illumination light 74 is used to illuminate the fundus in conjunction with the acquisition of images by the imaging sensor 32. In some embodiments, the illumination light 74 may also be used in conjunction with the spectroscopy module, as explained further below. As will be readily understood by one skilled in the art, the expression “operationally coupled” refers to any configuration allowing the illumination of the fundus by the electromagnetic radiation generated by the non-coherent source when performing an acquisition using the associated module.

In some implementations, the non-coherent source 72 is a component of an illumination system 70 designed in view of a particular application.

The expression “non-coherent” source is understood to refer to sources emitting light waves which have an amplitude and phase fluctuating randomly or quasi-randomly in space and time. In some implementations, the non-coherent source 72 may include one or more LED (Light Emitting Diode) emitters. The LED emitters of a given non-coherent source 22 may have similar optical properties or different complementary optical properties selected in order to obtain the desired optical properties of the illumination light 74 once combined. It will be readily understood that numerous other variants of light sources such as lasers, OLEDs, fluorescent, incandescent, tungsten, and other light bulbs may be used in alternative embodiments. The expression “illumination light” is used herein to refer to electromagnetic radiation suitable for projection into the eye 21 of a patient and for inducing, producing or otherwise generating return light 75 which can yield information of interest on the fundus 30 of the patient's eye 21 upon suitable analysis. It will be readily understood that the term “light” is not considered limited to the visible portion of the electromagnetic spectrum. The illumination light 24 preferably has a broadband spectral profile encompassing all the wavelengths of interest for the spectral analysis which the system is configured to perform. In some variants, the illumination light may be white light. In other variants, the illumination light 24 may have a spectral profile designed in view of the field of use of the multimodal device 20. In yet another set of variants, the illumination light may have any other suitable spectral profile as dictated by one or more factors such as the optical properties of the patient's eye, the availability of light sources, the nature and characteristics of the spectral analysis to be performed, etc.

It will be readily understood that the illumination module 70 may be embodied by any suitable collection or optical components and accompanying structural, mechanical, electrical or other features collaborating to bring the illumination light 74 from the non-coherent source 72 to the patient's eye 22 with the desired optical characteristics. The components of the illumination module 70 may redirect, focus, collimate, filter or otherwise act on light in a variety of fashions. One skilled in the art will readily understand that a multitude of designs may provide such a result. For example, in some variants, one or more optical fibers may be used to carry the illumination light 74 at least partially from the non-coherent source 72 towards the fundus 24. It will be further understood that the non-coherent source 72 may be provided either separately or integrally to the illumination module 70. In one example, the illumination module 70 may include a light port (not shown) configured to receive the illumination light 74 directly or indirectly from the non-coherent source 72. In some instances, the illumination module 70 has a design specific to a fundus camera and is preferably used in order for the imaging sensor 32 to receive a sufficient intensity return light from the fundus for the desired application. Such a design may for example includes one or more masks, filters, hole mirrors and the like configured to minimize reflections from interfaces other than the eye fundus.

In the illustrated configuration, by way of example only, the illumination module 70 includes a holed mirror 76 positioned at an angle along the imaging path 34. The holed mirror 38 has a central hole 77 aligned with the imaging path 34. Preferably, the non-coherent source 72 is positioned orthogonally to the eye of the patent 22, and the holed mirror 76 makes an angle (shown as 45° in the figure by way of example) with respect to the optical axis of the illumination light 74. In alternative embodiments, the illumination module 70 may include one or more optical components having variable transmission and reflection properties, for example a mirror designed to have a low reflectivity in the center and a high reflectivity around this center.

The illumination module 70 may further include beam-shaping optics projecting the illumination light 74 from the non-coherent source 72 onto the holed mirror 76 for reflection towards the fundus 24 of the patient's eye 22. The beam shaping optics may include one or more optical components interacting with the illumination light 74. In the illustrated embodiment, the beam-shaping optics includes a collimating lens 78 disposed along the optical path between the non-coherent source 72 to the holed mirror 76. A mask 80 may be positioned between the collimating lens 78 holed mirror 76. The mask 80 is optically aligned with the central hole 77 of the holed mirror 76 and is sized to block the center of the illumination beam 74. This configuration provides the illumination light 24 with an annulus shape so that it is reflected on the holed mirror 76. A screen 82 may be further provided between the collimating lens 78 and the mask 80 to prevent retroreflected light from the mask 80 from reaching the non-coherent source 72. The objective lens 62 and any other relevant optics may be provided between the holed mirror 76 and the eye of the patient 22.

### Spectroscopy Module

The spectroscopy module 40 includes a spectral analyzer 42 configured to receive electromagnetic radiation from the second region 26 of the fundus 24 through a spectral path 44 and to produce therefrom a spectral analysis of said second region.

In some variants, the spectral analyser 42 may be embodied by any suitable device or combination of devices allowing an analysis of light as a function of wavelength. The spectral analyser 42 may for example be embodied by an optical spectrometer. As known to one skilled in the art, optical spectrometers decompose incoming light according to its wavelength, typically using light refraction (e.g. using a prism) or light diffraction (using a diffraction grating), and include a detector measuring the distributed intensity of the decomposed light, for example a CCD or a CMOS sensor. The spectral analyser 42 may include a computer or processor programmed with instructions to analyse the detected light spectrum in accordance with predetermined parameters. In some embodiments the spectral analyser 42 may be grating-based. It will however be readily understood that a variety of other configurations and structural components may be used without departing from the scope of the present description. By way of example, the spectral analyser 42 may include at least one dispersive element such as grating in reflection or transmission or a prism. In other implementations, the spectral analyser 42 may include a plurality of individual photodetectors each detecting a specific or a range of specific wavelengths, for example using associated filters. In another example, the individual photodetectors may be spectrally separated by another dichroic/polarization/fiber circulator beam-splitting arrangement, similar to the one used to separate the imaging and spectral analysis light paths.

In some implementations, the spectroscopy module 40 is configured to provide a spectroreflectometric analysis. In the context of the present application, the expression “spectroreflectometric” is generally used as a contraction of the terms “spectral” and “reflectometric” in reference to techniques related to spectral reflectometry. As readily understood by those skilled in the art, reflectometry refers to the use of reflected light or other electromagnetic waves to analyse the properties of a medium. Light, such as for example the illumination beam 74 from the illumination module 70, is projected towards the medium and the interactions of the light wavefront with the medium interface leads to the generation of return light having optical properties affected by the medium. In spectral reflectometry, a spectral analysis of the return light, that is, an analysis of the properties of the return light as a function of its wavelength profile, is used to obtain or deduce information about the medium and its composition. Diffuse reflectance spectrum can be used in ophthalmologic contexts to measure oxygen levels in the fundus of the eye of a patient or to evaluate other biomarkers. Those compounds and their fluctuations in time are indicative of metabolism and pathophysiology. As mentioned above, oxygen levels are assessed through the presence of oxyhemoglobin which has a characteristic light absorbance pattern. Similarly, the concentration of deoxyhemoglobin, and carboxyhemoglobin (related to the levels of carbon dioxide present) can be determined based on their respective light absorbance patterns. These parameters and their regulation are indicative of metabolism, responses to stress and stimuli and, potentially, pathophysiologies. It will however be understood that other molecules and phenomena may also be studied, such as for example fluorescence, inasmuch as they lead to alterations in the spectral profiles of reflected light. It will also be understood that the spectral analysis may be performed for different regions of the fundus of the patient's eye or on features present on the fundus. In other implementations, the spectral analysis may be performed on other portions of the eye such as the conjunctiva. In some implementations, the spectral module 40 may alternatively or additionally be configured to perform a photoluminescence analysis, for example a fluorescence analysis, of a patient's eye. The expression photoluminescence generally refers to the emission of light following the absorption of photons by a medium, while the expression “fluorescence” is understood to refer to a particular type of photoluminescence resulting from a singlet-singlet electronic relaxation. In some implementations, the multimodal device 20 may be used in the context of the analysis of autofluorescence from the fundus of the patient's eye. “Autofluorescence” is a special case of fluorescence where the light emission originates from molecules native to the medium under study. In other applications, the analysed fluorescence may originate from fluorophores artificially added to the medium as markers. Fluorescence imaging is a useful procedure for clinicians as a tool for diagnostic of various ocular pathologies. By way of example, it is known to perform an autofluorescence imaging of lipofuscin granules (LGs). LGs will tend to accumulate in the retinal pigment epithelium of the eye of patients with hereditary diseases and, in particular, in age-related macular degeneration (AMD). Quantifying the LGs at a specific location in the retina can provide better diagnoses and evaluation of the severity of diseases such as AMD. Fluorescence analysis may be performed for different regions of the fundus of the patient's eye or on other features present on the fundus. In other implementations, fluorescence analysis may be performed on other portions of the eye such as the conjunctiva.

In photoluminescence-based embodiments, the spectroscopy module 40 may include an excitation light source 47 for generating an excitation light beam 49 whose spectral contents include one or more excitation wavelengths selected to excite the generation of photoluminescent light at one or more emission wavelengths in the fundus 24. The excitation light beam 49 is coupled to the spectral path 44 for projecting on the second region of the fundus of the patient's eye 22, which defines the analysis area. The excitation beam 49 may be absorbed by fluorescent compounds or the like present in the retina, exciting a singlet state of the fluorescent compounds. The excited compound will then thermally lose energy and emit light at the emission wavelength, which has a lower energy than the excitation photon. An optical filter 41 having a low light transmissivity at the excitation wavelength or wavelengths and a high light transmissivity at the emission wavelength or wavelengths may be coupled to the spectral analyser 42. The optical filter 41 is preferably a long-pass filter if the emission wavelength is longer than the excitation wavelength. In some implementations where the emission wavelength is shorter than the excitation wavelength, a low-pass filter may be used. In other variants, a bandpass filter or a notch rejection filter configured to allow light at the emission wavelength through and block light at the excitation wavelength may be used.

In some embodiments, the multimodal device 20 may be configured to include a pointer mode in which such that a pointer light beam 48 is projected on the fundus 24 of the patient's eye 22 at the second region 26 of the fundus, defining the analysis area of the spectral analyser 42. In the illustrated embodiment of FIG. 3B, the multimodal device 20 for example may include a pointer light source 43 optically coupled to the spectral path 44 by a coupler 45 so that the pointer light beam 48 can counter propagate along the spectral path 44 towards the primary beamsplitter 90. The pointer light source 43 may be embodied by any light source generating a pointer light beam suitable for pointing functionalities. In one example, the pointer light source 43 may be a laser diode. The pointer light beam 48 may have any wavelength or spectral contents safe for the eye of the patient and within the detection range of the imaging sensor. The pointer light source 43 may for example emit in the visible or near infrared spectral ranges. The operation of the pointer light source 43 are explained in further details in international patent application WO2019/109186, the entire contents of which is incorporated herein by reference.

### OCT Module

Still referring to FIGS. 3A and 3B, the OCT module 50 comprises an OCT detector 52, as mentioned above, configured to receive electromagnetic radiation from the third region 27 of the fundus 24 through the OCT path 54 and to produce therefrom depth-resolved information on said third region.

As known to those skilled in the art, OCT uses low-coherence interferometry to detect the difference in travelling delay of light rays to obtain the corresponding difference in depth distances. In typical OCT configurations, a low-coherence light source generates a light beam which is split in two halves (or another useful fraction). One half of the light is directed to a mirror at a known position on a reference arm, while the other half is directed to a sample arm and impinged on the sample (such as the fundus of a patient's eye), where it is scattered and reflects off tissue structures. Light from the reference and sample arms is then recombined to form an interference pattern, which is detected by a light detector.

The information encoded in the interference pattern relates the optical reflectance along the depth of the tissue under the point location at which the light beam impinges on the sample, creating what is known as an A-scan. By linearly scanning the location on the sample where this light beam impinges on the sample, a cross-sectional image of the sample is obtained, which is referred to in the art as a B-scan. Obtaining B-scans across several planes of the sample provides a 3D representation of the sample.

In some implementations wherein the third region is an analysis spot on the fundus of the patient's eye, the obtained depth-resolved image is an A-scan under this spot. In more typical implementations, the third region is a B-scan of a full or partial line across the first region, for example a B-scan across the optic nerve head of the fundus.

In some applications, Time Domain OCT (TD-OCT) is used. When using the TD-OCT technique, the location of each reflection is encoded in the time information relating the position of a moving reference mirror to the location of the reflection. Other applications may use Spectral Domain OCT (SD-OCT). In such variants, all information is acquired in a single axial scan through the tissue simultaneously by evaluating the frequency spectrum of the interference between the reflected light and a stationary reference mirror. Other OCT configurations, such as Swept-Source-OCT (SS-OCT).

Referring more particularly to FIG. 3B, there is shown an example of a multimodal device 20 using an OCT module 50 configured according to a SD-OCT approach. In such a configuration, the OCT module 50 includes an OCT light source 53 emitting an OCT light beam 51, typically having a short coherence length, low temporal coherence and a large optical bandwidth. The OCT light source 53 may for example be embodied by, but is not limited to, broadband light sources with short temporal coherence lengths or swept laser sources both in the visible and NIR wavelengths range. In some embodiments, the OCT light source 53 may emit light source in the NIR region, hence its usage can either be simultaneous or sequential to imaging and spectroscopic acquisition. The OCT light source 53 is optically coupled to the OCT path so that a first portion of the OCT light beam 51a travels along the OCT path 54 towards the fundus of the patient's eye. As explained above, light from the OCT light beam is scattered and reflected by the fundus in accordance with the optical reflectance along the depth of the tissue. The resulting light travelling back along the OCT path 54 towards the OCT module 50 defines an OCT analysis portion 58 of return light 75 from the fundus of the patient's eye.

The OCT module further includes a reference arm 56 which received a second portion of the OCT light beam 51 from the OCT light source 53. The reference arm 56 may form an optical delay line inducing a variable delay in the second portion of the OCT light beam 51b received directly from the OCT light source 53. By way of example, the reference arm 56 may include an OCT mirror 57 positioned across a path of the second portion of the OCT light beam. In operation, the length of the optical delay line is scanned longitudinally, for example by translating the OCT mirror 57 using a translation mechanism 59 such as a piezo transducer, to gain information on different longitudinal positions in the fundus. The delayed second portion of the OCT light beam 51b′ from the reference arm 56 and the return light 51a′ resulting from the interaction of the first portion of the OCT light beam 51a with the fundus are combined so as to form an interference pattern in which the degree of constructive interference will vary according to the delay induced by the reference arm 56. The OCT detector 52 therefore acquires a signal that is the resulting interference of the light having travelled through the reference arm and the light back-scattered from the fundus. In this example, the OCT detector 52 may for example be embodied by a photodetector, a photomultiplier tube or the like.

An OCT coupler 55 is provided to divide the OCT light beam 51 into the first and second portions 51a and 51b and direct the first portion to the OCT light path and the second portion to the reference arm 56, and to combine the return light 51a′ and 51b′ from both the OCT light path and the reference arm and direct the resulting interference pattern to the OCT detector 52. The OCT coupler may for example be embodied by a fiber coupler or by any optical arrangement having the desired beam dividing and combining characteristics, as well known in the art.

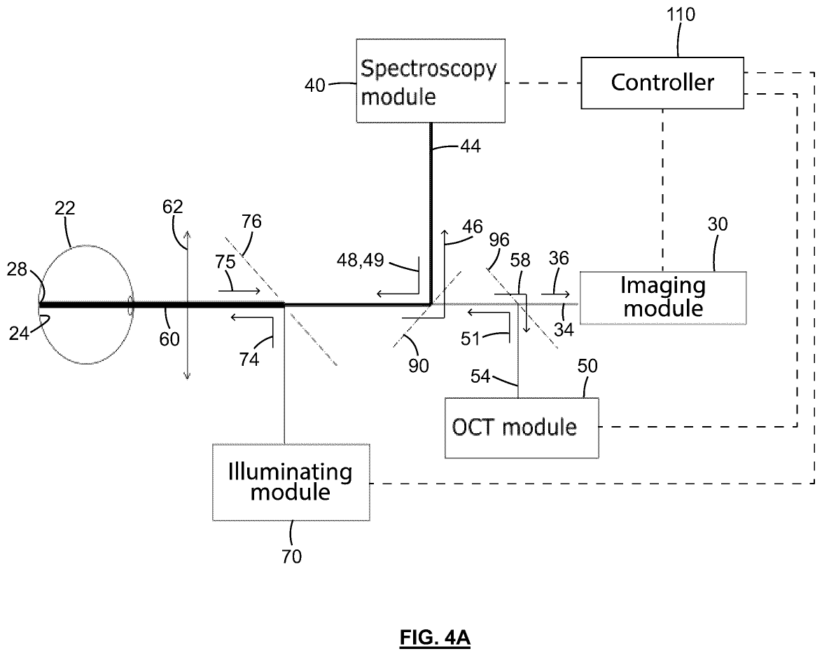

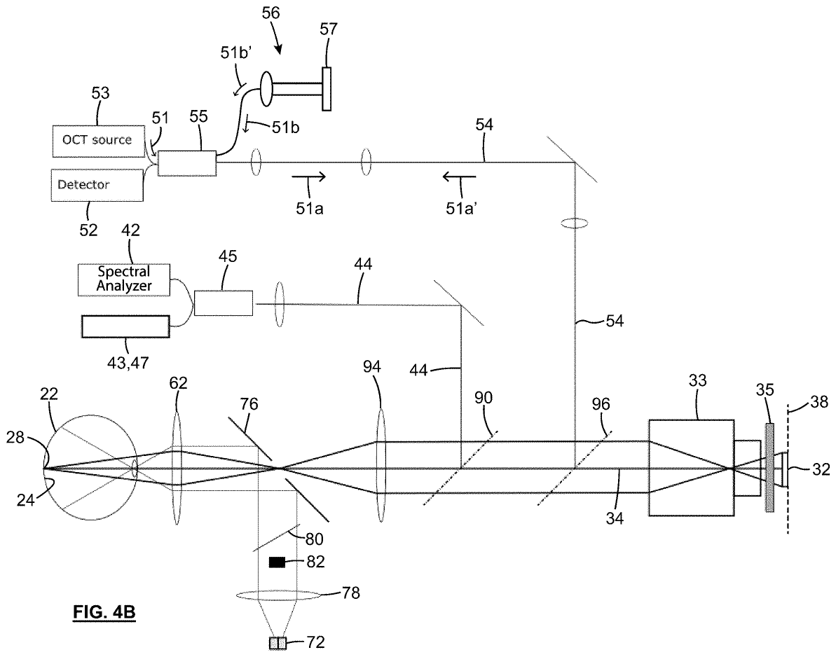

Referring to FIG. 4B, in another implementation the OCT module may have a SD-OCT configuration. In this variant, the OCT mirror 57 is fixed and the OCT detector 52 allows a spectrally resolved detection of the interference pattern, and may for example be embodied by a spectrometer or other device decomposing the signal according to wavelength (or optical frequency). Each wavelength corresponds to a different depth within the fundus which can be obtained through a Fourier transform of the detected signal.

In some implementations, the multimodal device 20 may further include a scanning module 64 provided along the OCT path 54 and configured to move the first portion of the OCT light beam 51a across the surface of the fundus of the patient's eye. This scanning of the first portion of the OCT light beam provides for depth information to be obtained from different point locations in the fundus, which collectively define the third region. In the illustrated example of FIG. 3B, the scanning module 54 may be embodied by a pair of pivotable mirrors 102 and 104. In some implementations, the scanning module may be configured to enable a displacement of both the second region 26 and the third region 27 (e.g., within the first imaging region). In other words, the scanning module allows moving the field of view of the spectral analyzer 42 and the field of view of the OCT detector 52 within or outside of the image acquired by the imaging sensor 32. In some embodiments, the multimodal device may include more than one scanning devices within the scanning module, configured to scan the first region and the second region separately.

### Multimodal Integration of the Imaging, Spectroscopy and Oct Modules

Referring particularly to FIG. 3B, the multimodal device 20 may further include a primary beamsplitter 90. In this embodiment, the primary beamsplitter 90 is positioned at an intersection of the imaging path 34, the spectral path 44 and the OCT path 54. It will be understood that the primary beamsplitter 90 defines an extremity of the common light path segment 60 opposite the fundus 24 of the patient's eye. In the illustrated embodiment, the primary beamsplitter 90 is configured to direct a spectral analysis portion 46 and an OCT analysis portion 58 of the return light 75 towards the spectral analyzer 42 and the OCT detector 52, and to direct a remainder of the return light 75, embodying an imaging portion 36, to the imaging path 34. It will be readily understood that although in the illustrated variant the primary beamsplitter 90 is configured and arranged to transmit through the imaging portion 36 of the return light 75 and reflect the spectral and OCT analysis portions 46, 58 of the return light 75, in other variants it may be configured and arranged to reflect the imaging portion 36 towards the imaging sensor 32, and transmit through the spectral and OCT analysis portions 46, 58 towards the spectral analyser 42 and OCT detector 52.

The primary beamsplitter 90 may operate according to any one of various principles to separate the light impinging thereon according to different portions. In one embodiment, the primary beamsplitter 90 is a dichroic beamsplitter. As well known in the art, dichroic optical components affect light according to its spectral characteristics. The primary beamsplitter 90 may have a spectral transmission profile tailored to the operation of the multimode system 20. As one skilled in the art will readily understand, selecting the spectral profiles are the primary beamsplitter 90 and of the light sources preferably aims to optimize the collected signals.

In some implementations, a focussing lens 94 may be positioned between the holed mirror 76 and the primary beamsplitter 90. In some embodiments, the focussing lens 94 may be mounted on a suitable translation actuator allowing its displacement along the imaging path 34. Such a movement displaces the imaging plane 38 to compensate for refractive index variations in the eye of different patients. In other variants, the focussing lens 94 may have a variable focus and may be adjustable by different means.

The imaging portion 36 of the return light 75 travels along the imaging path 34 until it reaches the imaging sensor 32 for detection. Of course, numerous optical components could be provided along the imaging path 34 to collimate, focus, filter, redirect or otherwise affect the imaging portion 36 prior to reaching the imaging sensor 32. Beamshaping optics 33 are indicated as a black box representation of such components in the illustrated configuration of FIG. 3B. This illustrated embodiment also includes an optional wavelength filter 35 placed between the imaging sensor 32 and the patient's eye 22 to enable enhance discrimination of certain spectral information on the fundus 24.

The spectral and OCT analysis portions 46, 58 of the return light 75 are deviated by the primary beamsplitter 90 along a common segment of the spectral analysis path 44 and the OCT path 54 and is incident on a secondary beamsplitter 96. The secondary beamsplitter 96 is configured to separate the spectral analysis portion 46 and the OCT analysis portion 58 along different light paths. In some implementations, the secondary beamsplitter 96 is a dichroic beamsplitter positioned and having a spectral transmission profile tailored such that light at wavelengths of interest to the spectral analysis and the OCT analysis are respectively directed towards the spectral analyser 42 and the OCT detector 52. In other embodiments, in particular for variants where the spectral analyzer and the OCT detector share the same wavelength range, the secondary beamsplitter 96 may separate light according to other characteristics than its spectral content, such as intensity ratios or polarization state.

In some implementations, the scanning module 64 may be configured to shift the position of the second region 26 over the fundus 24 of the patient's eye without impact on the first region 25. The scanning module 64 may include shiftable optics such as one or more steerable mirrors. In the illustrated example of FIG. 3B, a pair of pivotable mirrors 102 and 104 are provided between the primary beamsplitter 90 and the secondary beamsplitter 96. A pivoting of the pivotable mirrors 102 and 104 can be used to change the incidence angle between the light travelling along the common segment of the spectral path 44 and the OCT path 54 and the primary beamsplitter 90. In some implementations, the pivotable mirrors 102, 104 are movable in both the phi and the theta directions and are used to change the angle of incidence of the pointer light beam 48 and OCT light beam 51 onto the primary beamsplitter 90. The direction of displacement of the first mirror 102 is opposite to the one of the second mirror 104 in order to keep the position of the pointer light beam 48 and OCT light beam 51 on the primary beamsplitter 90 and to only change the angle of incidence. Other spot shifting schemes can be envisioned by one skilled in the art. For example, a gimbal mirror may be used in place of the second mirror 104 jointly with a pair of lenses used in a 4f configuration.

The multimodal device 20 preferably includes a controller 110 providing control signals to the active components of the imaging module 30, spectral module 40 and OCT module 50, as well as the illumination module 70 and scanning module 64. In some implementations, the controller 110 is responsible for triggering the different components of the device with the desired frequency, synchronization and timing. The components to be triggered may include the light emitting sources, the light sensing devices, and the motors. For the imaging module, the non-coherent source is preferably triggered at the same time as the CCD or CMOS imaging device to acquire an image of the first region. For the spectroscopy module, the spectrometer has to be triggered at the same time as the non-coherent source used for imaging to acquire a spectrum in the visible range at the same time. Reference can for example be made to international patent application WO2019/109186 for more details on this synchronization sequence.

In some embodiments, the controller 110 is configured to synchronize the lateral displacement in the third region 27, hence the field of view of the OCT detector 52, via the scanning module 64, with the longitudinal displacement of the OCT reference arm 56, the generation of the OCT light beam 51 and the activation of the OCT detector 52. In typical implementations, the OCT detector 52 acquires light from the OCT portion of the return light 58 for a very small amount of time and associates an intensity value to the detected light. This value is recorded and allocated 3D spatial information. An image reconstruction technique may be used, such as for example reconstructing the different measured intensity associated with coordinates into a grayscale representation.

To prevent cross-talk between the spectral analyzer 42 and the OCT detector 52, various optical elements of the multimodal device 20 may be selected so as to separate the different signals either via wavelength or temporally, via sequential acquisition. The image acquired by the imaging module 30 may be used as the reference to direct the OCT and/or spectral acquisitions.

Synchronization of the imaging module 30, the spectral module 40 and the OCT module 50 is preferably performed using precise and high frequency electronic hardware and firmware in the associated devices and the controller 110. In some implementations, the imaging module 30 and the spectral module 40 both make use of fundus illumination via the non-coherent light source 72 of the illumination module 70, which is preferably adapted to operate according to a similar level of precision and high frequency as the other modules. In typical implementations, the reference arm 56 of the OCT module 50 and the scanning module 64 are preferably adapted to provide both depth information and to scan the third region 27 over the fundus 22 within the same time required for the spectral acquisition by the spectral module 40. By way of example, in some implementations the imaging sensor 32 is a CCD sensor such as the Sony ICX274AQ or a CMOS sensor such as the Sony Pregius, the spectral analyzer 42 is a spectrometer that uses a CCD or a CMOS sensor, such as products from Ocean Optics and Hamamatsu, in other cases the spectrometer section of the system could be multiple detectors combined with optical filters, and the OCT detector 52 is a photomultiplier tube (PMT) such as the ones supplied by Hamamatsu that convert light into an electrical signal or an avalanche photodiode such as the ones provided by Hamamatsu that rely on internal gain produced by the application of a reverse voltage to obtain a better signal-to-noise-ratio.

It will readily be understood that other configurations and instruments may alternatively be used to combine the imaging module, the spectral module and the OCT module in a same multimodal device. Referring to FIGS. 4A and 4B, such an alternative configuration is shown, by way of example. In the illustrated variant, The OCT module 50 is coupled to the imaging path 34 between the imaging module 30 and the primary beamsplitter 90. The primary beamsplitter 90 is configured to direct the spectral analysis portion 46 of the return light 75 towards the spectral analyzer 42, and allowing a remainder of the return light 75 through. The secondary beamsplitter 96 is here positioned downstream from the primary beamsplitter 90 so as to intersect the remainder of the return light 75 and deviate the OCT portion 58 of the return light 75 towards the OCT module 50. The imaging portion 36 of the return light 75 is transmitted through the secondary beamsplitter 96 and directed towards the imaging module 30.

### Operation Scenarios

In a typical use scenario, the image acquired using the imaging module 30 is used to direct the acquisitions associated with the spectroscopy module 40 and the OCT module 50. Based on the information visible on the image, a user may either define a region of interest as a specific location on the fundus 24, define multiple regions of interest on the fundus 24 or define a scanning pattern to perform on the fundus 24. The selected region of interest, defined by the user, can depend on unexpected features present on the fundus 24, or may simply be region-based protocol, e.g. taking measurement in the optic nerve head and/or in the macular region. In some implementations, the region of interest is the region from where additional acquisition will be performed and correspond to the second region 26. Displacement of the pointer light beam 48 may be used to align of the region of interest with the region of acquisition from the spectroscopy module. This principle has already been described in patent application WO2019/109186.

Once the region of interest is selected, both the spectral acquisition and the OCT acquisition may be performed. In some implementations, the user may either select the acquisition of spectral information from this region or a 3D acquisition. By way of example, the spectroscopy module 40 enables the acquisition of diffuse reflectance spectra or a generated photoluminescent light at an emission wavelength differing from the excitation wavelength. The technical details for acquiring such information has been described in WO 2020/243842, the entire contents of which is incorporated herein by reference. Spectral acquisition may rely on the use of the same broadband non-coherent source 72 used for illumination by the imaging module 30. Then, within this specific region of spectral acquisition, the OCT light beam 51 from the OCT light source (e.g., a partially coherent light source, e.g., a coherent light source) 53 is scanned to provide cross-sectional OCT imaging simultaneously to the spectral acquisition.

Referring to FIGS. 5A to 5D, a typical workflow and expected outputs are represented. FIG. 5A shows an image of the fundus of a patient's eye acquired by the imaging module. FIG. 5B shows the selection of a region of interest (ROI) on the fundus image. FIGS. 5C and 5D respectively show a spectral analysis and cross-sectional imaging of the selected ROI. Using jointly a incoherent light source (e.g. a visible light source and/or an near-infrared (NIR) light source) for spectroscopy and fundus imaging and a partially coherent light source (e.g. a visible light source and/or an NIR light source) for OCT, within a shared field of view on the eye fundus enables new insights from the tissue. In some embodiments, spectroscopy and OCT could be performed using the partially coherent source. In some embodiments, spectroscopy, fundus imaging and OCT could be performed using the partially coherent source. Structural information (e.g., from OCT) can be deployed to improve molecular measurements (e.g., from spectroscopy) by providing inputs (e.g., thicknesses or retinal layers) to theoretical models of light propagation. Such inputs can be used to adjust and/or calibrate measurements in order to provide more precise and complete information about the tissue. Similarly, in some embodiments, the combined structural and molecular information (e.g., OCT, fundus images and spectral analysis) may be deployed in combination using modern analytical methods, such as machine learning, deep learning, artificial intelligence to further expand the information of acquired. In some embodiments, information acquired by the multimodal system may be combined with patient metadata to perform assessments (e.g., for patient health).

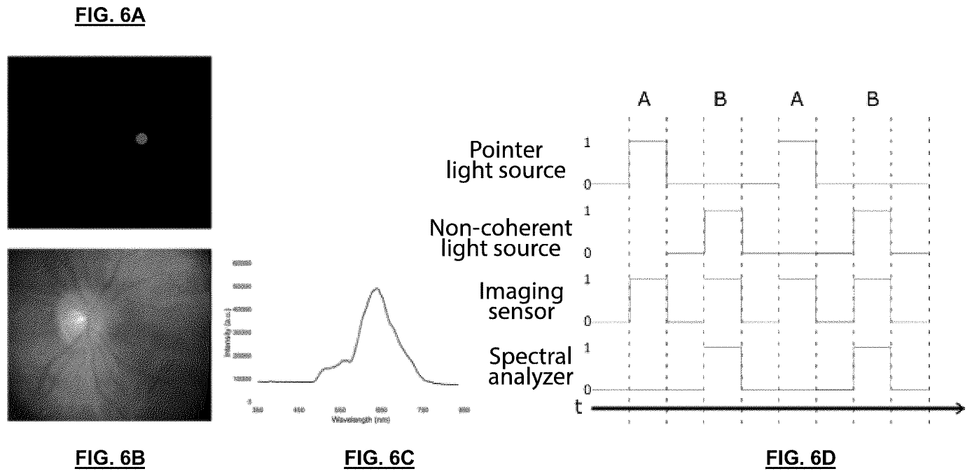

FIG. 6D shows an example of a timing diagram for sending control signals to the pointer light source 43, the non-coherent light source 72, the imaging sensor 32 and the spectral analyzer 42 of the multimodal device illustrated in FIG. 3B. In accordance with some implementations, the controller 110 may be configured to operate the device in a pointer mode, obtaining the image of FIG. 6A, and an imaging and spectral acquisition mode, obtaining the image of FIG. 6B and the spectrum of FIG. 6C. The images of FIGS. 6A and 6B may be combined to allow a user to visualize the position of the pointer light beam on the fundus. In some implementations, the pointer mode and spectral acquisition modes may be operated alternatively, whereas in other implementations they may be operated concurrently. The control signals may have various profiles and it will be understood that the signals shown in FIG. 6D are provided for illustrative purposes only. For example, while the control signal for the imaging sensor is shown as an AC signal in FIG. 6D, in other implementations a DC signal may be used.

In the pointer mode, the pointer light source 43 and the imaging sensor 32 are turned on, whereas the non-coherent light source 72 is turned off. As a result, an image of the pointer light beam 49 is acquired by the imaging sensor. This can also be done by selecting a proper light source for pointing that does not interfere with the collected signal and the image. In the spectral acquisition mode, the pointer light source 43 is turned off while the non-coherent light source 72, the imaging sensor 32 and the spectral analyzer 42 are turned on. An image of the first region 25 of the eye fundus is obtained as well as a spectral analysis of the second region 26 targeted by the pointer light beam.

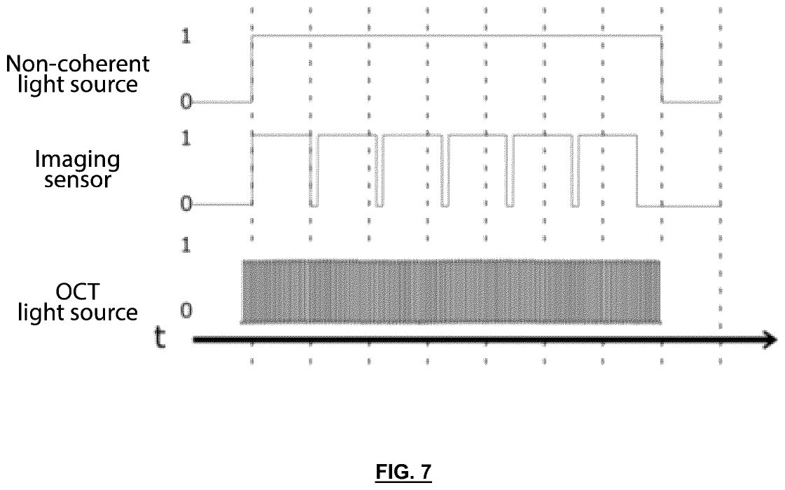

FIG. 7 shows a timing diagram for sending control signals to the non-coherent source 72, the imaging sensor 32 and the OCT light source 53. This enables the acquisition of a cross-section image of the eye fundus. A NIR light source is used to illuminate the fundus and enables the imaging device to capture images of the fundus to position the OCT acquisition. Following the alignment, the OCT acquisition begins, which implies high frequency triggers for both the OCT source and the OCT detector. Frequency of operation for a spectral domain OCT is typically between 20 kHz and 70 KHz A-scan/s; however, in some embodiments of the current disclosure, A-scan rates may be greater than 100 kHz. An A-scan corresponds to a single-depth profile produced by an OCT system. A B-scan is the scanning of OCT beam in a single direction (e.g., nasal/temporal on a retina) to create a slice (e.g., a 2D image). A C-scan is the scanning of an OCT beam in 2 dimensions (e.g., nasal/temporal and dorsal/ventral on a retina, e.g., circularly) to create a 3D image volume. In some embodiments of the current disclosure, the third imaging region (e.g., the OCT beam location) may be scanned to perform B-Scans or C-Scans.

In summary, embodiments of the multimodal device described herein and equivalents thereof offer a one-stop tool for analysis of the fundus of a patient's eye and provide new, multimodal information on the state of the patient's eye. In typical implementations, the device enables one to acquire a large field of view, for example more than 20 degrees, from the eye fundus. Using this, a clinician can select a region of particular interest. This region can be selected based on pigmentation changes or abnormalities, such as the presence of drusen or cotton-wool spots, or based on structural regions, such as the fovea or the optic nerve head. From this region, with a smaller field of view, (for example less than 3 degrees), spectral or photoluminescence information from the medium is acquired. This information can provide valuable information on metabolism, the pathophysiology of different illnesses and conditions, or the efficacy of administered treatments. Simultaneously to the spectral or photoluminescence analysis, additional structural information is acquired by scanning the region with a coherent light source. Those two complementary information, photoluminescence analysis and internal structure details via OCT, provide a full portrait of a given region and mutually bonify each other. The cross-sectional information will help identify the region from where the reflected light is coming from and the spectral or photoluminescence information will provide information about the optical properties of the tissue that will shine a new light on the cross-sectional details.

Of course, numerous modifications could be made to the embodiments described above without departing from the scope of the invention as defined in the appended claims.

1. A multimodal device for performing an assessment of the fundus of a patient's eye, the multimodal device comprising:
an imaging module comprising an imaging sensor having an angular field of view encompassing a first region of the fundus, the imaging sensor configured to receive electromagnetic radiation from said first region through an imaging path and to produce therefrom an-image representative of said first region;
a spectroscopy module comprising a spectral analyzer having an angular field of view encompassing a second region of the fundus, the second region being located within the first region, the spectral analyzer configured to receive electromagnetic radiation from the second region of the fundus through a spectral path and to produce therefrom a spectral analysis of said second region; and
an OCT module comprising an OCT detector having an angular field of view encompassing a third region of the fundus, the third region being at least partially located within the first region, the OCT detector configured to receive electromagnetic radiation from the third region of the fundus through an OCT path and to produce therefrom depth-resolved information on said third region;
wherein the imaging path, the spectral path and the OCT path have a common light path segment leading to the fundus of the patient's eye.

2. The multimodal device according to claim 1, further comprising an illumination module comprising a non-coherent light source generating illumination light and optically coupled to the common light path segment for illuminating the first region of the fundus of the patient's eye with said illuminating light.

3. The multimodal device according to claim 2, wherein the spectroscopy module comprises an excitation light source generating an excitation light beam coupled to the spectral path for projecting on the second region of the fundus of the patient's eye.

4. The multimodal device according to claim 2, further comprising a pointer light source generating a pointer light beam coupled to the spectral path for projection on the second region of the fundus of the patient's eye.

5. The multimodal device according to claim 1, wherein the spectral analysis produced by the spectral module comprises a spectroreflectometric analysis.

6. The multimodal device according to claim 1, wherein the spectral analysis produced by the spectral module comprises a photoluminescence analysis.

7. The multimodal device according to claim 1, wherein the OCT module further comprises:
an OCT light source configured to generate an OCT light beam having a short coherence length;
a reference arm; and
an OCT coupler configured to divide the OCT light beam into a first portion and a second portion and optically coupled to the OCT light path and to the reference arm;
whereby the first portion of the OCT light beam is coupled into the OCT light path for projection on the third region of the fundus of the patient's eye, and the second portion of the OCT light beam is coupled into the reference arm.

8. The multimodal device according to claim 7, wherein the OCT module has a Time-Domain OCT configuration.

9. The multimodal device according to claim 7, wherein the OCT module has a Spectral Domain OCT configuration.

10. The multimodal device according to claim 8, further comprising a scanning module provided along the OCT path and configured to move the first portion of the OCT light beam across the fundus of the patient's eye, thereby defining the third region.

11. The multimodal device according to claim 10, wherein the spectral path and the OCT path have a shared spectral and OCT segment, and the scanning module is provided within the shared spectral and OCT segment.

12. The multimodal device according to claim 11, comprising:
a primary beamsplitter connecting the imaging path and the shared spectral and OCT segment, the common light path segment extending between the primary beamsplitter and the fundus of the patient's eye; and
a secondary beamsplitter connecting the spectral path and the OCT path, the shared spectral and OCT segment extending between the secondary beamsplitter and the primary beamsplitter.

13. The multimodal device according to claim 1, comprising:
a primary beamsplitter coupling the spectral path and the imaging path, the common light path segment extending between the primary beamsplitter and the fundus of the patient's eye; and
a secondary beamsplitter coupling the OCT light path to the imaging light path downstream of the primary beamsplitter.

14. The multimodal device according to claim 17, further comprising a controller generating control signals for controlling active components of the imaging module, spectral module, OCT module, illumination module and scanning module.

15. The multimodal device according to claim 14, wherein the controller is configured to operate the multimodal device to:
a) acquire the image representative of said first region using the imaging module;
b) identifying the second region and third region based on said image;
c) acquire the spectral analysis of said second region; and
d) acquire the depth-resolved information on the third region.

16. The multimodal device according to claim 14, wherein the controller is configured to operate the multimodal device in:
an illumination mode wherein the controller operates the illumination light source to project said illumination light towards the patient's eye, the imaging device to obtain the image representative of said first region of the fundus and the spectral analyser to obtain said spectral analysis of said second region of the fundus; and
an OCT mode wherein the controller operates the OCT light source, OCT detector and scanning module to obtain the depth-resolved image of the third region of the fundus.

17. The multimodal device according to claim 10, further comprising an illumination module comprising a non-coherent light source generating illumination light and optically coupled to the common light path segment for illuminating the first region of the fundus of the patient's eye with said illuminating light.
